SEQUENCE LISTING (6 sequences)

SEQ ID NO: 1 - DNA, 24 bp, Artificial
  mutant TREX1 crystallization oligonucleotide
tcacgtgctgacgtcagcacgacg

SEQ ID NO: 2 - DNA, 24 bp, Artificial
  PCR primer
cctgctgctactcattaccccatc

SEQ ID NO: 3 - DNA, 25 bp, Artificial
  PCR primer
cctactccatgtcagggagagagga

SEQ ID NO: 4 - DNA, 20 bp, Artificial
  TREX1 genotype sequencing primer
acagcatcgctgccctaaag

SEQ ID NO: 5 - DNA, 21 bp, Artificial
  PCR primer
agggacagggcagaccaagaa

SEQ ID NO: 6 - DNA, 20 bp, Artificial
  PCR primer
accggagtggacccgtcatt

(19) United States
(12) Patent Application Publication
(10) Pub. No.: US 2019/0021294 A1
(43) Pub. Date: Jan. 24, 2019

(54) TRANSGENIC ANIMALS EXPRESSING MUTANT TREX1 PROTEIN USEFUL AS A MODEL OF AUTOIMMUNE DISEASE

(71) Applicant: Wake Forest University Health Sciences, Winston-Salem, NC (US)

(72) Inventor: Fred W. Perrino, Winston-Salem, NC (US)

(21) Appl. No.: 16/069,035

(22) PCT Filed: Jan. 13, 2017
(86) PCT No.: PCT/US2017/013285

§ 371 Date: Jul. 10, 2018

Related U.S. Application Data
(60) Provisional application No. 62/279,298, filed on Jan. 15, 2016.

(51) Int. Cl. A01K 67/027

(52) U.S. Cl. CPC A01K 67/0275; A01K 2267/0325; A01K 2227/105; A01K 2217/072

(57) ABSTRACT

Provided herein is a recombinant or transgenic non-human mammal comprising a nucleic acid encoding a mutant three-prime exonuclease 1 (three prime repair exonuclease 1; TREX1), and in particular aspects the mammal expresses the mutant Trex1 protein. The non-human mammal is useful for identifying candidate compounds for the treatment of autoimmune disease (in human or animal, typically mammalian) subjects. Another aspect of the invention is, accordingly, a method for identifying candidate compounds for the treatment of autoimmune disease or disorder comprising: providing the recombinant non-human mammal; administering a test substance to the recombinant non-human mammal; and determining whether said test substance reduces at least one indicia of autoimmune disease in said mammal, wherein a reduction in said at least one indicia indicates said test substance is a candidate compound for the treatment of autoimmune disease.

### RELATED APPLICATIONS

This application claims priority to U.S. Provisional Patent Application Ser. No. 62/279,298 filed Jan. 15, 2016, the disclosure of which is hereby incorporated by reference in its entirety.

### STATEMENT OF GOVERNMENT SUPPORT

This invention was made with Government Support under grant numbers RO1GM069962 05A1S1 and RO1GM069962 awarded by the National Institutes of Health. The United States Government has certain rights to this invention.

### BACKGROUND OF THE INVENTION

The TREX1 gene encodes a powerful DNA exonuclease (1-7). The amino terminal domain of the TREX1 enzyme contains all the structural elements for full exonuclease activity and the carboxy terminal region controls cellular trafficking to the perinuclear space (8-10). Mutations in TREX1 cause a spectrum of autoimmune disorders including Aicardi-Goutieres syndrome, familial chilblain lupus, retinal vasculopathy with cerebral leukodystrophy, and are associated with systemic lupus erythematosus (9, 11-19). The TREX1 disease-causing alleles locate to positions throughout the gene, exhibit dominant and recessive genetics, include inherited and de novo mutations, and cause varied effects on catalytic function and cellular localization. These genetic discoveries have established a causal relationship between TREX1 mutation and nucleic acid-mediated immune activation disease. The spectrum of TREX1-associated disease parallels the diverse effects on enzyme function and localization indicating multiple mechanisms of TREX1 dysfunction might explain the overlapping clinical symptoms related to failed DNA degradation and immune activation.

The TREX1 enzyme structure reveals the uniquely stable dimer that is relevant to its function and to disease mechanisms. The backbone contacts between the protomer β3-strands generate a stable, central antiparallel β-sheet that stretches the length of the dimer and an extensive hydrogen-bonding network of sidechain-sidechain, sidechain-backbone, and water-bridged contacts that coordinate residues across the TREX1 dimer interface (8, 20). TREX1 catalytic function depends on the dimeric structure with residues from one protomer contributing to DNA binding and degradation in the opposing protomer (21, 22). Some TREX1 disease-causing mutants exhibit complete loss of catalytic function while others exhibit altered cellular localization (8, 10). A subset of TREX1 catalytic mutants at amino acid positions Asp-18 and Asp-200 exhibit selectively dysfunctional activities on dsDNA. These mutations cause autosomal dominant disease by retaining DNA binding proficiency and blocking access to DNA 3′ termini for degradation by TREX1 WT enzyme (21, 23, 24). The TREX1 catalytic sites accommodate four nucleotides of ssDNA and additional structural elements are positioned adjacent to the active sites for potential DNA polynucleotide interactions.

The connection between failure to degrade DNA by TREX1 and immune activation was first made in the TREX1 null mouse that showed a dramatically reduced survival associated with inflammatory myocarditis (25). However, the origin and nature of the disease-driving DNA polynucleotides resulting from TREX1 deficiency have not been clearly established. One model posits that TREX1 acts in the SET complex to degrade genomic dsDNA during granzyme A-mediated cell death by rapidly degrading DNA from the 3′ ends generated by the NM23-H1 endonuclease (26). Two additional models propose that TREX1 prevents immune activation by degrading ssDNA, but these models differ on the possible source of offending DNA polynucleotide. In TREX1-deficient cells there is an accumulation of ssDNA fragments within the cytoplasm proposed, in one model, to be generated from failed processing of aberrant replication intermediates that result in chronic activation of the DNA damage response pathway (27, 28). Another model proposes the source of accumulating ssDNA in TREX1-deficient cells to be derived from unrestrained endogenous retroelement replication leading to activation of the cytosolic DNA sensing cGAS-STING pathway (29-33). This concept is also supported by the participation of TREX1 in degradation of HIV-derived cytosolic DNA (34). Thus, disparate concepts on the DNA polynucleotide driving immune activation in TREX1 deficiency have been proposed, and it is possible that the robust TREX1 exonuclease participates in multiple DNA degradation pathways.

### SUMMARY OF THE INVENTION

We present here structural and in vivo data supporting the concept that Trex 1 degradation of dsDNA is critical to prevent immune activation.

An aspect of the invention is, accordingly, a recombinant non-human mammal. The mammal comprises a recombinant or transgenic nucleic acid encoding a mutant three prime exonuclease 1 (three prime repair exonuclease 1; TREX1) and in particular aspects the mammal expresses the mutant Trex1 protein.

The non-human mammal is useful for identifying candidate compounds for the treatment of autoimmune disease (in human or animal, typically mammalian) subjects. Another aspect of the invention is, accordingly, a method for identifying candidate compounds for the treatment of autoimmune disease or disorder comprising: providing the recombinant non-human mammal; administering a test substance to the recombinant non-human mammal; and determining whether said test substance reduces at least one indicia of autoimmune disease in said mammal, wherein a reduction in said at least one indicia indicates said test substance is a candidate compound for the treatment of autoimmune disease.

Autoimmune diseases which the non-human mammal is useful for identifying candidate compounds for the treatment thereof include, but are not limited to: Aicardi-Goutieres syndrome (AGS); alopecia areata; familial chilblain lupus (FCL); multiple sclerosis (MS); polymyalgia rheumatica; retinal vasculopathy with cerebral leukodystrophy; rheumatoid arthritis (RA); STING-associated vasculopathy with onset in infancy (SAVI); Sjögren's syndrome, schleroderma; systemic lupus erythmatosus (SLE); celiac disease; ankylosing spondylitis; T1D; temporal arteritis; and vasculitis. In some embodiments, the autoimmune disease or disorder is selected from the group consisting of: systemic lupus erythmatosus (SLE); Aicardi-Goutieres syndrome (AGS); familial chilblain lupus (FCL); and retinal vasculopathy with cerebral leukodystrophy.

In some embodiments, the test substance binds to and/or inhibits cyclic GMP-AMP synthase (cGAS).

In some embodiments, the test substance binds to and/or inhibits stimulator of interferon genes (STING).

In some embodiments, the the test substance binds to and/or inhibits TANK-binding kinase 1 (TBK1) and/or interferon regulatory factor 3 (IRF3).

The present invention is explained in greater detail in the drawings herein and the specification set forth below. The disclosure of all United States patent references cited herein are to be incorporated herein by reference in their entirety.

### BRIEF DESCRIPTION OF THE DRAWINGS

FIGS. 1A-1F. The TREX1 D18N allele is expressed, processed, and translated. (FIG. 1A) The TREX1 single exon gene is shown with the two donor (GT) and acceptor (AG) sequences. Transcripts 1 (t1) and 2 (t2) are generated from the two donor sites. (FIG. 1B), TREX1 cDNA products were generated from TREX1^{WT/D18N} mouse liver RNA using forward and reverse primers positioned as shown in (FIG. 1A). Reactions containing reverse transcriptase (RT) generated t1 and t2. The genomic (g) TREX1 sequence was generated in reactions ±RT. (FIG. 1C), Sequencing of t1, t2, and g bands in (FIG. 1B) demonstrates appropriate t1 and t2 splicing (left) and expression of TREX1 D18N and WT alleles (right). (FIG. 1D), TREX1 expression in mouse tissues (6 animals of each genotype). SG, salivary gland; CLN, cervical lymph node. (FIG. 1E), Western blot of TREX1 protein (ng) purified from mouse tissues. The position of migration of TREX1 is indicated. The band identified as TREX1 D18N was excised from a gel and confirmed by mass spec analysis (FIG. 1F), Activity assay of TREX1 protein (pg) purified from mouse tissues. *p<0.05; **p<0.001.

FIGS. 2A-2H. Trex1^{D18N/D18N} mice have a shortened lifespan and an autoimmune phenotype. (FIG. 2A), Survival curve. (FIG. 2B), Spleen weight (3-8 animals/genotype). (FIG. 2C), Splenocyte cell counts (10 animals/genotype). (FIG. 2D), Spleen H&E. Bar=100 μm. (FIG. 2E), Heart H&E. Bar=100 μm. (FIG. 2F), Kidney H&E. Bar=50 μm. *indicates tubular proteinosis. (FIG. 2G), Splenic vessel H&E. Bar=50 μm. (FIG. 2H), Histology scores for inflammation. SG, salivary gland. (FIG. 2I), Histology scores for splenic lymphoid hyperplasia, vasculitis, and renal disease. ND, not detected. *p<0.05; **p<0.001; bars represent the mean and error bars represent s.e.m.

FIGS. 3A-311. TREX1^{D18N/D18N} mice produce α-dsDNA antibodies, form glomerular immunocomplexes, and have activated adaptive immune responses. (FIG. 3A), Total IgG ELISA (10-12 animals/genotype). (FIG. 3B), Total ot-dsDNA antibody ELISA (10-12 animals/genotype). (FIG. 3C), Immunofluorescence (IF) for IgG in frozen kidney sections. Bar=50 μm. Splenocytes were labeled for (FIG. 3D), CD4, CD8, and B220; (FIG. 3E), CD138; (FIG. 3F), CD4, CD69 and CD62L; (FIG. 3G), CD8, CD69 and

CD62L; or (FIG. 3H), CD4, CD25, and FoxP3, and enumerated by flow cytometry. *p<0.05; **p<0.001; bars represent means and error bars represent s.e.m.

FIG. 4. Replacement of the TREX1 WT allele with the TREX1 D18N allele. The R1 129 mouse TREX1 genomic region (˜10kb) flanked by Sad sites (lower) and the vector used for targeted homologous recombination (upper) are shown (Boxes, exons; lines, introns; squiggle lines, mouse genomic sequences flanking the region of homology; D18N codon, point mutation introduced into the vector TREX1 coding exon and C→T change in TREX1 5′UTR; NEO, neomycin resistance gene; DT-A⁻, diphtheria toxin A gene; LoxP, recognition site for Cre recombinase; PCR1-7, PCR primers). The linearized TREX1 D18N vector was electroporated into XSV1 ES cells (of a 129S6/SvEvTac origin) and DT-A⁻G418-resistant colonies were selected for screening using PCR1/PCR2 and PCR3/PCR4 to identify candidate ES cell clones for targeted homologous recombination. Both PCR3 and PCR4 contain a 3′ terminal mispair unless the ES clone contains the targeted TREX1 D18N mutant allele. PCR positive clones for both reactions with the expected size PCR products had integrated the NEO linked vector into the TREX1 locus. A third PCR using PCRS/PCR6 recovers a 1.4 kb genomic fragment and sequencing confirmed targeting of the Trex1 mutant allele and the appropriate heterozygosity corresponding to the presence of both the WT and mutant alleles in the ES cell clones. Positive D18N ES clones were expanded, mouse C57BL/6NTac blastocysts were injected, chimeric mice were generated, and germline transmission of the TREX1 D18N allele from chimeras was confirmed by sequencing of tail DNA. Chimeras were bred to 129-Tg(Prm-Cre)58 Og/J mice (Jackson Laboratory). The transgene in this strain is comprised of the mouse protamine 1 promoter and the Cre recombinase coding sequence and mediate the efficient recombination of a Cre target transgene in the male germ line, but not in other tissues; therefore two rounds of breeding were used: breeding of chimeras to the Cre-deleter females (N1 generation) and then—their heterozygous male progeny to 129SvEvTac females (N2 generation). Cre deletion of the NEO cassette was confirmed by PCR.

FIG. 5. Endogenous TREX1^{D18N/D18N} protein. Extracts were prepared from TREX1 WT and TREX1^{D18N/D18N} mouse tissues and loaded onto separate ssDNA-cellulose columns, washed, and eluted proteins were fractionated by SDS-PAGE. TREX1 protein was detected by immunoblotting as described below. Lane 1, Bio-Rad mol. wt. prestained standards; Lane 2, pure recombinant mouse TREX1 1-242 (24ng) (1); Lane 3, HEK 293T whole cell lysate transfected with pEBB-TREX1 (1-314) N-HA (N terminal HA tag) (Orebaugh C D, et al. J Biol Chem 288(40):28881-28892 (2013)); Lane 4, HEK 293T whole cell lysate transfected with empty pEBB plasmid; Lane 5, blank; Lanes 6-8, increased amounts of ssDNA cellulose elution from mouse TREX1 WT sample; Lane 9, blank; and Lanes 10-12, increased amounts of ssDNA cellulose elution from mouse TREX1 D18N sample. The band indicated as TREX1 was confirmed by mass spec analysis. Protein eluted from the ssDNA cellulose column and the same mol. wt. standards (Lanes 1-4) were fractionated by SDS-PAGE and stained with Coomassie Blue. The region of the lane predicted to contain the mouse TREX1 D18N protein, as determined by the size standards, was excised and the TREX1 D18N protein was detected in the sample by mass spec analysis.

### DETAILED DESCRIPTION OF ILLUSTRATIVE EMBODIMENTS

The TREX1 protein and TREX1 gene (e.g., the “wild type” TREX1 protein and gene) are known and described in, for example, U.S. Pat. No. 6,632,665 to Fred W. Perrino (human and mouse), the disclosure of which is incorporated herein by reference in its entirety. The Mus musculus TREX1 sequence is also described at NCBI Reference Sequence: NP_035767.4.

A mutant TREX1 protein or gene as described herein is one differing from the wild-type protein or gene by including at least one mutation, such as a substitution or deletion mutation.

Recombinant or transgenic non-human mammals containing a mutant TREX1 gene and expressing the same can be made in accordance with known techniques, or variations thereof that will be apparent to those skilled in the art. See, e.g., A. Doyle et al., The construction of transgenic and gene knockout/knockin mouse models of human disease; Transgenic Res. 21, 327-349 (2012); T. Bayer, Transgenic mouse model expressing amyloid B4-42 peptide, U.S. Pat. No. 9,204,623 (2015); F. Zhang, CRISPR-CAS systems and methods for altering expression of gene products, U.S. Pat. No. 8,697,359 (2014); L. McLonlogue et al., Transgenic rodents harboring APP allele having Swedish mutation, U.S. Pat. No. 6,586,656 (2003); A. Beaudet et al., Non-human animal having predefined allele of a cellular adhesion gene, U.S. Pat. No. 5,602,307 (1997).

As noted above, a first aspect of the invention is a recombinant non-human mammal. The mammal comprises a recombinant or transgenic nucleic acid (e.g., heterologous nucleic acid) encoding a mutant three prime exonuclease 1 (three prime repair exonuclease 1; TREX1). The mammal preferably expresses the mutant TREX1 protein.

In some embodiments, the mammal preferably expresses auto-antibodies to double stranded DNA.

In some embodiments, the mammal preferably exhibits systemic inflammation, lymphoid hyperplasia, vasculitis, and/or kidney disease (e.g., deposition of immune complexes in the kidneys).

In some embodiments, the recombinant nucleic acid is operatively associated with an endogenous TREX1 promoter.

In some embodiments, the mammal is a rodent, such as a mouse or rat, or a primate, such as a monkey.

In some embodiments, the mammal is homozygous for the mutant TREX1; in other embodiments, the mammal is heterozygous for the mutant TREX1.

The mutant TREX1 preferably contains a (or “at least one”) substitution mutation.

In some embodiments the substitution mutation is a D18N substitution mutation. See Grieves et al., PNAS vol 112, no. 16, 5117-5122 (Apr. 21, 2015). In other embodiments, the substitution mutation is not a D18N substitution mutation.

In some embodiments, the substitution mutation is a D18H substitution mutation. In some embodiments, the substitution mutation is a D200N, D200H, D200A, R114H, T303P, Y305C, P290L, or G306A substitution mutation. In some embodiments, the substitution mutation is a T13N, T32R, K66R, L92Q, R97H, V122A, R128H, P132A, A158V, L162P, R185C, H195Q, H195Y, E198K, V201D, V201N, D220G, A223T, G227S, R240S, or A247P substitution mutation.

The non-human mammal is useful for identifying candidate compounds for the treatment of an autoimmune disease or disorder (in human or animal, typically mammalian) subjects. In general, the method may be carried out, by: (a) providing a transgenic non-human mammal as described herein; (b) administering a test substance to the non-human mammal; and (c) determining whether the test substance reduces at least one indicia of autoimmune disease or disorder in the mammal, wherein a reduction in the at least one indicia indicates the test substance is a candidate compound for the treatment of the autoimmune disease or disorder.

In some embodiments of the foregoing, the determining step may be carried out by: (a) measuring the at least one indicia in the mammal before the administering step, measuring the at least one indicia in the mammal after the administering step, and comparing the two; and/or (b) comparing the at least one indicia in the mammal after the administering step with the at least one indicia in a corresponding transgenic non-human mammal that has not been administered the test substance. Examples of suitable indicia include, but are not limited to, expression of auto-antibodies to double stranded DNA, systemic inflammation, lymphoid hyperplasia, vasculitis, kidney disease, or a combination thereof (and including cell markers of any thereof).

Autoimmune diseases or disorders for which the non-human mammal of the present invention may be used to identify candidate compounds for the treatment of said autoimmune disease or disorder include, but are not limited to: Aicardi-Goutieres syndrome (AGS); alopecia areata; amyotrophic lateral sclerosis (ALS); ankylosing spondylitis; familial chilblain lupus (FCL); glaucoma; multiple sclerosis; (MS); polymyalgia rheumatica; retinal vasculopathy with cerebral leukodystrophy; rheumatoid arthritis (RA); schlerodeima; Sjögren's syndrome; spinocerebellar ataxia, autosomal recessive, 23 (SCAR23); STING-associated vasculopathy with onset in infancy (SAVI); systemic lupus erythmatosus (SLE); celiac disease; T1D; temporal arteritis; and vasculitis. In some embodiments, the autoimmune diseases or disorders include systemic lupus erythmatosus (SLE) and related disorders, for example, Aicardi-Goutieres syndrome (AGS), familial chilblain lupus (FCL), and retinal vasculopathy with cerebral leukodystrophy.

The non-human mammal of the present invention harbors one or more mutations in the TREX1 gene, which may result in the dysfunction of the encoded Trex1 exonuclease. Without wishing to be bound to any one theory or mechanism related to an autoimmune disease or disorder, candidate compounds/test substances may be examined for their ability to reduce at least one indicia of an autoimmune disease or disorder. The indicia may be associated, for example, with a disease phenotype resulting from or linked to the dysfunction of the TREX1 exonuclease. In some embodiments, this disease phenotype may include an increase in the activity or stimulation of cyclic GMP-AMP synthase (cGAS). Cyclic GMP-AMP (cGAMP) functions as a second messenger that binds and activates the stimulator of interferon genes (STING) transmembrane protein to induce IFN and cytokine expression by triggering phosphorylation/activation of interferon regulatory factor 3 (IRF3) and NF-κB transcription factors via TANK-binding kinase 1 (TBK1), which phosphorylated/activated transcription factors in turn promote transcription of inflammatory genes such as, for example, IFN-β. See, e.g., Ablasser et al., “TREX1 Deficiency Triggers Cell-Autonomous Immunity in a cGAS-Dependent Manner,” J Immunology 2014: 5993-5997; Gao et al., “Activation of cyclic GMP-AMP synthase by self-DNA causes auto-immune diseases,” PNAS 2015: E5699-E5705.

TANK-binding kinase 1 (TBK1) and IkappaB kinase epsilon (IKKε) have been validated as novel drug targets, with applications in the treatment of cancer, a variety of inflammatory diseases (including rheumatoid arthritis, COPD and psoriasis) and obesity. Diseases associated with TBK1 also include amyotrophic lateral sclerosis (ALS), frontotemporal dementia, glaucoma, etc.

Accordingly, in some embodiments, candidate compounds to reduce at least one indicia of an autoimmune disease or disorder include candidate compounds/test substances for the ability to bind to and/or inhibit cGAS, or inhibit cGAS activation or stimulation of STING. In other embodiments, candidate compounds to reduce at least one indicia of an autoimmune disease or disorder include candidate compounds/test substances for the ability to bind to and/or inhibit STING, or bind to and/or inhibit TBK1 and/or IRF3. In some embodiments, the candidate compounds/test substances may inhibit the binding and/or interaction between STING and cGAMP. In other embodiments, the candidate compounds/test substances may inhibit STING activation of IRF3 through TBK1. In yet other embodiments, the candidate compounds/test substances may inhibit STING activation of NF-κB. See, e.g., US 2016/0068560 to Patel et al. In yet other embodiments, the candidate compounds/test substances may bind to and/or inhibit IkappaB kinase epsilon (IKKε). In yet other embodiments, the candidate compounds/test substances may bind to and/or inhibit tumor necrosis factor receptor-associated factor 1 (TRAF1).

The nature of the candidate compounds/test substances is not particularly limited. In some embodiments, the candidate compounds/test substances may be small molecules, for example, compounds under the molecular weight of 900 daltons. In other embodiments, the candidate compounds/test substances may be macromolecules, for example, nucleic acids or nucleic acid mimetics, peptides, polypeptides, proteins and/or antibodies.

The present invention is explained in greater detail in the following non-limiting Examples.

### EXPERIMENTAL

The dominant negative effects of TREX1 D18N in the heterozygous genotype of individuals affected with familial chilblain lupus were revealed in the DNA degradation properties of the hetero- and homodimer forms of TREX1 likely to exist in cells of these individuals. The TREX1 WT homodimers and the WT protomer within heterodimers containing a D18N mutant protomer are fully functional when degrading ssDNA polynucleotides (13). In contrast, TREX1 heterodimers and homodimers containing a D18N mutant protomer are inactive on dsDNA and block the dsDNA degradation activity of TREX1 WT enzyme, providing a genetic and mechanistic explanation linking dysfunctional TREX1 and human disease phenotype (21, 23, 24). The selective catalytic inactivity of TREX1 D18N on dsDNA indicates a significant difference in the interactions of TREX1 with ss- and dsDNA likely linked to DNA unwinding.

The TREX1 D18N-dsDNA structure reveals a novel unwinding mechanism. To elucidate the mechanisms of TREX1 D18N dominant mutant dysfunction, we determined the crystal structure of the TREX1 D18N mutant protein in complex with dsDNA. The structure of the TREX1 D18N-dsDNA complex reveals a novel dsDNA unwinding mechanism that feeds a single-stranded terminus into the active site and exposes a defect in metal ion binding that contributes to catalytic inactivity. Similar to the DNA repair nucleases that share common elements while exhibiting individually unique mechanisms (35), there is little change in the core TREX1 structure while the flexible regions bind, melt, and reshape the dsDNA to position ssDNA that is specifically required for catalysis into the active site. Together, the tight protein-DNA interactions coupled with a catalytically inactive protein points to the biological dysfunction that connects TREX1 dominant mutants with disease.

TREX1 uses a nucleic acid kinking mechanism for unwinding dsDNA to provide ssDNA substrate to the active site. The TREX1-dsDNA structure reveals two distinct DNA binding steps in its dsDNA unwinding mechanism. The protein-dsDNA complex was crystallized with two TREX1 dimers and two dsDNA helices in the asymmetric unit. Each dimer has a 3′ end of the DNA bound in one of the active sites with the 3′ end of the complementary strand bound in the active site of an adjacent dimer, creating a ‘beads on a string’ type lattice throughout the crystal (not shown). A comparison of the four active sites within the TREX1-dsDNA complexes in the asymmetric unit reveals two distinct binding conformations in the ends of the DNA helices (not shown), representing separate steps in an unwinding process necessary to provide ssDNA for insertion into the active site. Both conformations have the terminal 3′ nucleotide correctly positioned in the active site, and both induce a kink in the substrate strand of the DNA at the point of transition from ds- to ssDNA. There are also marked differences in the conformations of the bound DNA and the associated interactions with each protomer of the TREX1 dimer. A comparison of the TREX1 D18N protein with the wild-type TREX1 (pdbid 2OA8, (8)) reveals little overall change in the core protein structure with an overall rmsd between the two of 0.64 Å. Additionally, the positions of the conserved catalytic residues are well maintained.

The first conformation, described here as the ‘tight’ conformation, likely represents an early step in DNA unwinding. The TREX1 protein makes contacts with both strands of the DNA. The substrate strand is tethered to the protein through interactions with active site residues, while the phosphodiester backbone of the complementary strand straddles helix α5, making contacts with residues W218, H222 and R224 (not shown). The helix α5 acts as a wedge into the minor groove inducing a widening of the groove to about 16.5 Å at the point of strand separation. The four nucleotides at the end of the substrate strand are unpaired with no visible density for the last three on the complementary strand. The last two nucleotides on the substrate strand (C23-G24) are correctly positioned in the active site for hydrolysis of the 3′ terminus. Proximal to this, the DNA backbone is severely distorted with a ˜95° bend in the phosphodiester backbone between nucleotides G21 and C23. The nucleotide in the middle of this kink (A22) is rotated into the minor groove (not shown). The flipping of nucleotide A22 by TREX1 is facilitated by residues R128 and K160 directly adjacent to the active site. The hydrophobic face of the flipped base is stabilized by its position directly above the side chain of 1156. R128 also makes cation-pi stacking interactions with the final unpaired nucleotide

(C4) of the complementary strand. The importance of residue R128 in the DNA binding and base-flipping process is consistent with the previously identified TREX1 mutation of R128 to histidine in a patient with neurophychiatric Systemic Lupus Erythematosus (18).

A second, ‘loose’ conformation captures the enzyme after the base-flipping step (not shown). Here, nucleotide A22 on the substrate strand is rotated back into a base stacking orientation with its phosphate group making contact with the amide nitrogen of Y177 and side chain oxygen of S176. The kink in the DNA backbone is now translated down the strand between nucleotides A22 and C20. Nucleotide C4 on the complementary strand that is visible in the tight conformation is now disordered indicating an unwinding of the DNA helix by one base pair relative to the tight conformation. Residue R128 makes only a single hydrogen bond interaction with the base of nucleotide G21 of the substrate strand. The complementary strand of DNA is no longer making interactions with W218, H222 and R224, but instead contacts residue R174 allowing the DNA helix to lift above the protein and reducing the widening of the minor groove to about 13 Å (not shown). This loose DNA binding conformation may facilitate release of the DNA after hydrolysis, consistent with the nonprocessive nature of the Trex 1 enzyme. The inhibition of dsDNA degradation by the dominant TREX1 D18N mutant can be explained by the enzyme being trapped in an inactive complex, which combines extensive protein-nucleic acid interactions necessary for dsDNA unwinding with a catalytically deficient active site. The TREX1 D18N-dsDNA structure reveals a single magnesium ion in the active site coordinated by the mutated residue N18, and a phosphate oxygen of the terminal 3′ nucleotide. The second metal ion necessary for catalysis is absent (data not shown). The positions of the two terminal nucleotides of the substrate and the active site residues in both the tight and loose conformations superimpose on each other and also superimpose on a ssDNA substrate bound within a wild-type TREX1 active site (data not shown), indicating that the absence of the second divalent metal ion contributes to catalytic inactivity of this mutant. Identification of possible altered dsDNA binding that could contribute to failed catalysis in TREX1 D18N will require comparison to TREX1 WT-dsDNA that is not currently available. The selectively dysfunctional degradation of dsDNA by the dominant TREX1 D18N mutant directly points to dsDNA as an important cellular substrate for TREX1. As described in structural studies of the DNA repair enzymes FEN1 (36) MREII (37), and RNase T (38) in complexes with DNA substrates, the TREX1 D18N-dsDNA structure reveals a unique protein design that incorporates DNA bending, base flipping, and structural wedges allowing TREX1 to facilitate dsDNA degradation in cells. The TREX1 core is most similar to the RNase T that also requires ssDNA in the active site. However, the TREX1 dimer forms very differently from the RNase T dimer exposing the necessary structural elements in TREX1 to allow dsDNA unwinding that is not possible in the RNase T.

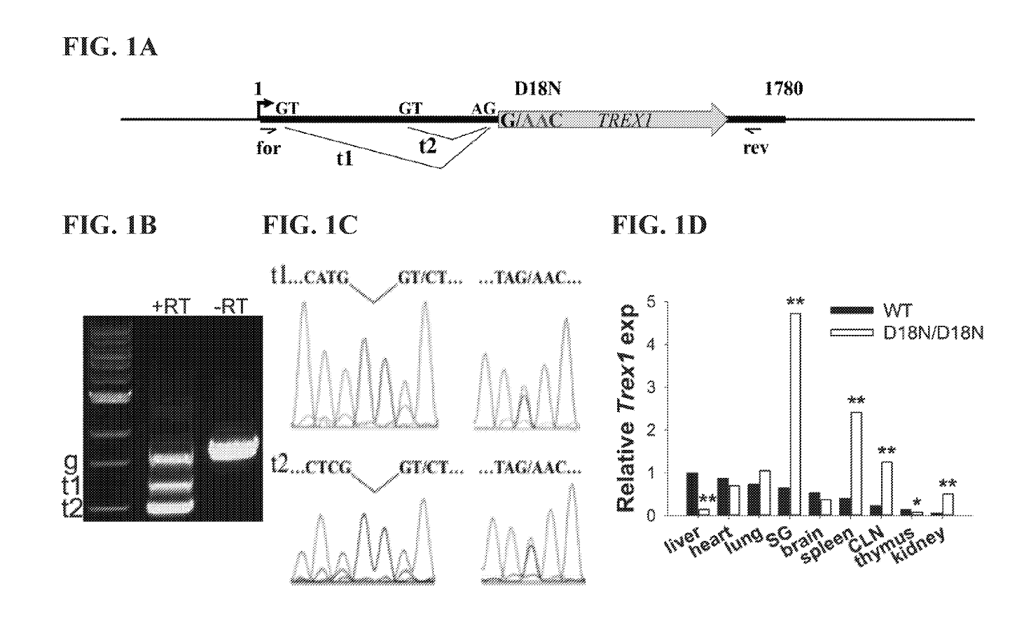

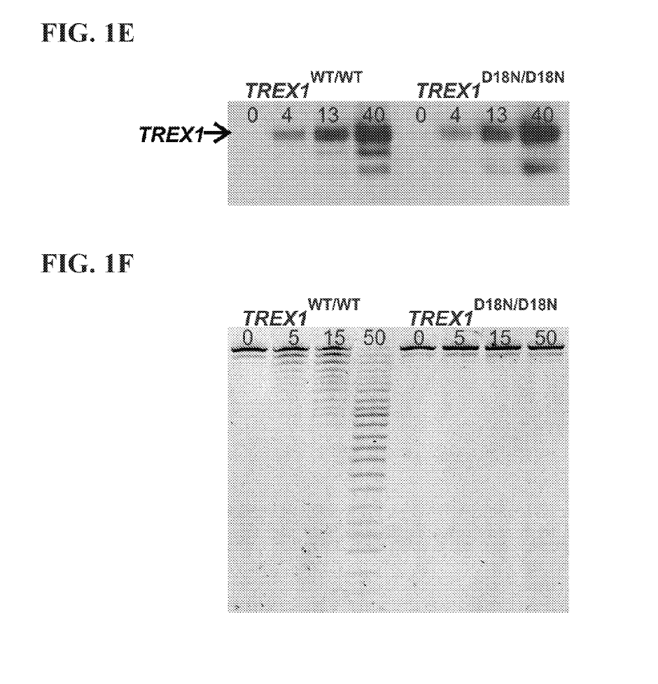

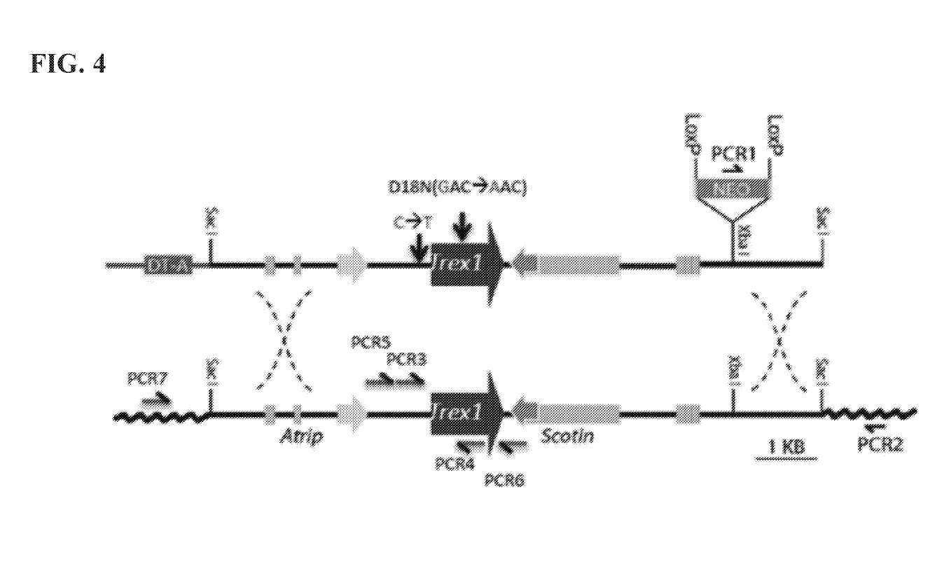

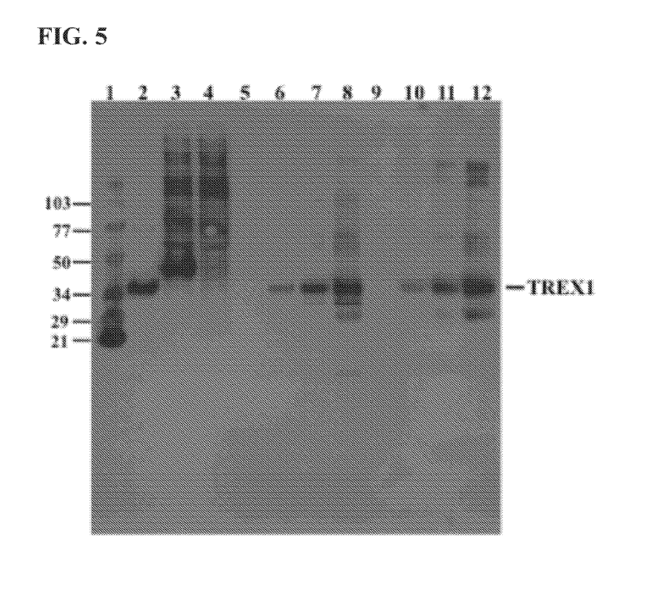

Spontaneous Lupus-like Inflammatory Disease in the TREX1 D18N Mouse. More than forty TREX1 mutant alleles have been identified that cause a variety of Lupus-like autoimmune diseases in humans, but the effects of these TREX1 mutations in mice are not known. To directly determine if dsDNA is a prominent autoantigen when the TREX1 D18N enzyme is present we tested the effects of the dominant TREX1 D18N mutation in vivo. A genetically precise mouse model that recapitulates the dysfunctional TREX1 pathway was generated using an allelic replacement strategy to express the mouse TREX1 D18N allele from its endogenous promoter that controls the level of expression in the appropriate genomic context on mouse chromosome 9 (See FIG. 4). The TREX1 D18N allele is expressed (FIG. 1A) and the transcript is processed identically to the TREX1 WT allele (FIG. 1B, 1C). The levels of TREX1 expression were quantified from selected tissues of animals at 8 wks. of age, prior to the histological detection of significant inflammation. Quantification of TREX1 expression in tissues from TREX1^{WT/WT} and TREX1^{D18N/D18N} mice (FIG. 1D) showed that expression varied in WT tissues and that levels were altered in some of the TREX1^{D18N/D18N} mice tissues as early as 8 wks. Interestingly, increased levels of expression in TREX1^{D18N/D18N} mice was detected in secondary lymphoid organs, which are sites of antigen accumulation, and in some of the tissues found later to be significantly inflamed in older TREX1^{D18N/D18N} mutant mice including the salivary gland and kidney. Previous work has shown that TREX1 is widely expressed by immune cells and can be induced in macrophages, B cells, and dendritic cells in vitro by proinflammatory stimuli (39). While it is possible that increased expression in TREX1^{D18N/D18N} mouse tissues reflects populations of macrophages and other inflammatory cells responding to proinflammatory stimuli, immune cell infiltration into tissues of TREX1^{D18N/D18N} mice does not occur to a significant extent at 8 wks. and not all tissues such as the heart and lung that exhibit significant inflammation later in life demonstrated increased TREX1 expression. To demonstrate the presence of TREX1 D18N protein in the TREX1^{D18N/D18N} mice, tissues from multiple organs were pooled from Trex1^{WT/WT} and Trex1^{D18N/D18N} mice, Trex1 enzyme was partially purified by ssDNA chromatography, and TREX1 protein was detected by immunoblotting (FIG. 1E) and by DNA exonuclease assay (FIG. 1F). Pooled tissues from TREX1^{WT/WT} and TREX1^{D18N/D18N} mice contained similar levels of TREX1 protein and exonuclease activity was abolished in TREX1^{D18N/D18N} tissues.

Expression of the mutant TREX1 D18N enzyme in TREX1^{D18N/D18N} mice results in a clinically distinct phenotype from that observed when TREX1 is completely absent, as is the case in the TREX1 knockout mice which do not breed successfully and succumb to cardiomyopathy at a median age of ˜10 weeks (25, 31). To determine the clinical phenotype of TREX1^{D18N/D18N} mice we monitored animals from 3 wk to 6 mo. of age. TREX1^{D18N/D18N} mice have similar growth characteristics (data not shown) and are typically clinically indistinguishable from WT littermates. TREX1^{D18N/D18N} mice mate successfully up to at least 6 months of age but have slightly smaller average litters than WT mice (4.6 vs 5.8, respectively) (Table 1).

TABLE 1
Mouse matings. The pups generated from WT, heterozygous, and
homozygous mice were enumerated to determine average litter size.

| | | | | Avg. # of |
|---|---|---|---|---|
| Mating type | Matings | Litters | Pups | pups/litter |
| Trex1^{WT/WT} × Trex1^{WT/WT} | 4 | 8 | 46 | 5.8 |
| Trex1^{WT/D18N} × Trex1^{WT/D18N} | 13 | 46 | 211 | 4.6* |
| Trex1^{D18N/D18N} × Trex1^{D18N/D18N} | 4 | 15 | 68 | 4.5* |

*p < 0.05

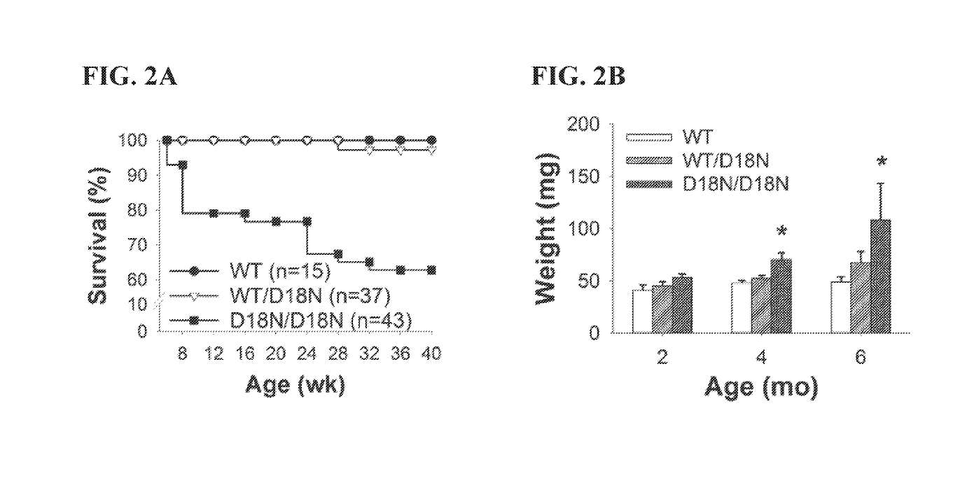

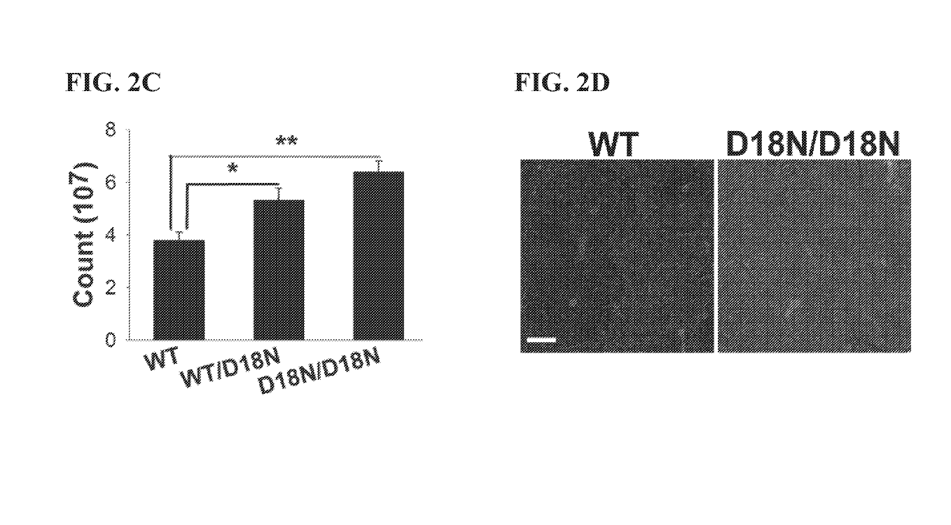

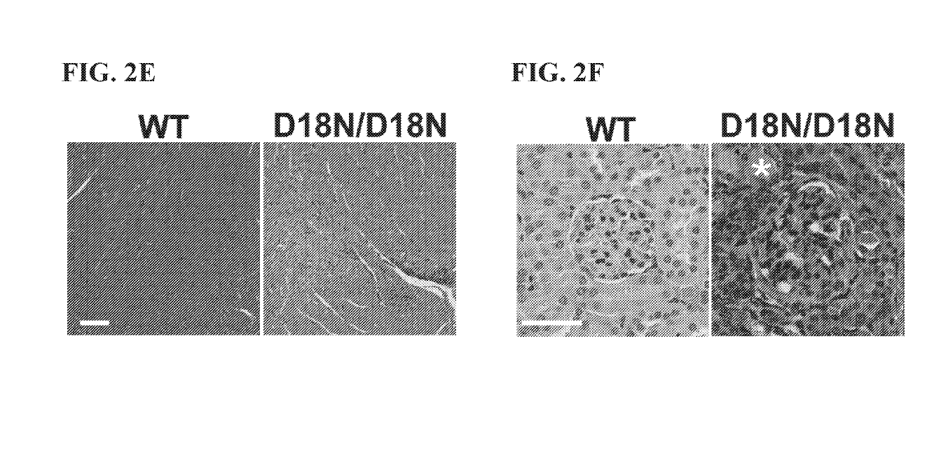

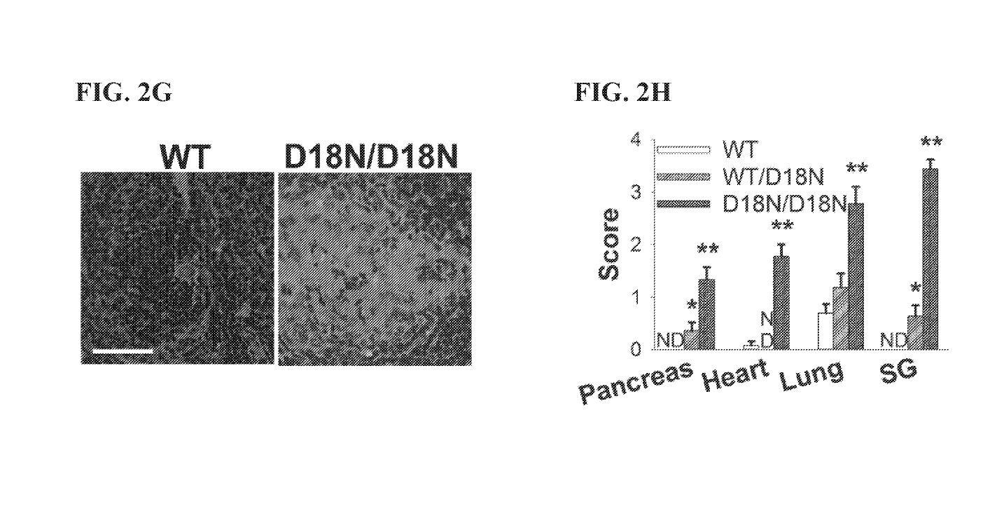

However, TREX1^{D18N/D18N} have a decreased life span with losses as early as 6 wks. of age (FIG. 2A). The effects of TREX1 D18N expression were similar in males and females and revealed when animals were sacrificed for phenotypic examination. The spleens of TREX1^{D18N/D18N} mice were enlarged as early as 4 mo. of age (FIG. 2B), which corresponded to increased splenic nucleated cell counts (FIG. 2C). Additionally, by 4-6 months of age lymph nodes were enlarged in the majority of TREX1^{D18N/D18N} mice, and the hearts of clinically healthy animals had variously sized regions of pallor and were often mildly to markedly enlarged with right or biventricular dilation. Of the TREX1^{D18N/D18N} mice that were euthanized due to clinical disease or died prior to the pre-determined sacrifice date, 87.5% had congestive heart failure characterized grossly by bicavitary effusions, chronic passive congestion of the liver, pulmonary atelectasis, and markedly enlarged hearts with atrial and ventricular dilation and often atrial thromboses. A TREX1^{D18N/D18N} mouse that needed to be euthanized had gross evidence of chronic kidney disease characterized by a shrunken and pitted appearance of the kidneys. Histologic analysis of tissues collected from TREX1^{D18N/D18N} mice revealed four major categories of consistently observed lesions that were the most severe at 6 mo. of age including lymphoid hyperplasia, inflammation, vasculitis, and kidney disease (FIGS. 2D-2G). Secondary lymphoid organs of TREX1^{D18N/D18N} mice had expanded lymphoid follicles and increased numbers of germinal centers, the site of B cell proliferation and maturation Inflammation of varying severity was present in the heart, lung, salivary gland, pancreas, and occasionally other organs including the lacrimal gland. The inflammatory infiltrates consisted of lymphocytes and large numbers of antibody-producing plasma cells Inflammatory cells in the lung and salivary gland were primarily surrounding bronchioles and ducts, respectively; suggesting that inhaled and ingested antigens might contribute to immune stimulation in TREX1^{D18N/D18N} mice. Vasculitis was present in both large and small caliber vessels of TREX1^{D18N/D18N} mice and was characterized by influx of inflammatory cells, medial necrosis, and disruption of the vessel wall by protein-rich deposits. Vasculitis is common in Lupus patients and the vascular lesions observed in TREX1^{D18N/D18N} mice mirror those seen in Lupus patients. Histopathology of kidney lesions indicated widespread to diffuse, global to segmental membranoproliferative glomerulonephritis and lymphoplasmacytic tubulointerstitial nephritis (FIG. 2H, 2I and Tables 2-5 below for histopathology scoring).

The multi-organ inflammation observed in TREX1^{D18N/D18N} mice is similar to the Lupus phenotype in humans where the lung, salivary gland, and heart are often targets and immune-complex glomerulonephritis is a common complication. Increased TREX1 expression in the salivary gland of 8 wk old TREX1^{D18N/D18N} mice was an unexpected finding and salivary glands were not significantly enlarged in older TREX1^{D18N/D18N} mice. While Familial Chilblain Lupus patients have systemic disease (16), there are no reports of clinical involvement of the salivary glands. Skin lesions including malar rashes and chilblains are a frequent finding in Lupus patients, but are not a feature of most spontaneous mouse models of Lupus (40-42) and likewise were not observed in TREX1^{D18N/D18N} mice. This is likely because skin lesions in Lupus patients are typically induced by environmental conditions including heat, moisture, and UV light that are not factors in conventionally housed rodents. Disease was most significant in TREX1 D18N homozygous animals, while heterozygous animals exhibited normal survival and rather minimal inflammatory differences compared to WT. This is notable because all known cases of TREX1 D18N-mediated disease in humans have been due to the heterozygous genotype. It is possible that environmental stimuli and viral or bacterial infection could exacerbate disease in heterozygous animals.

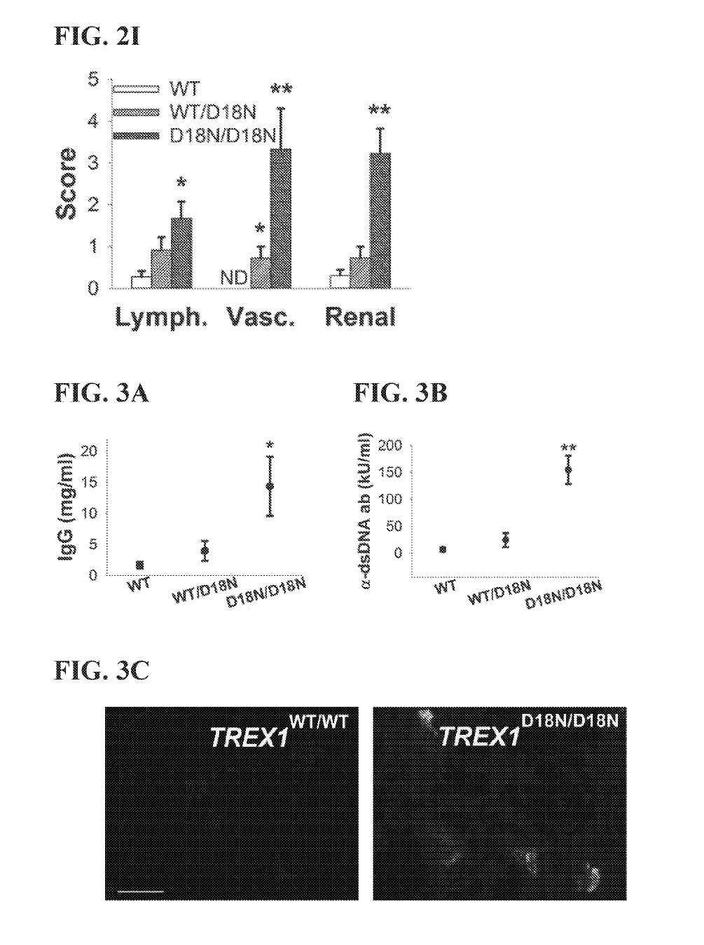

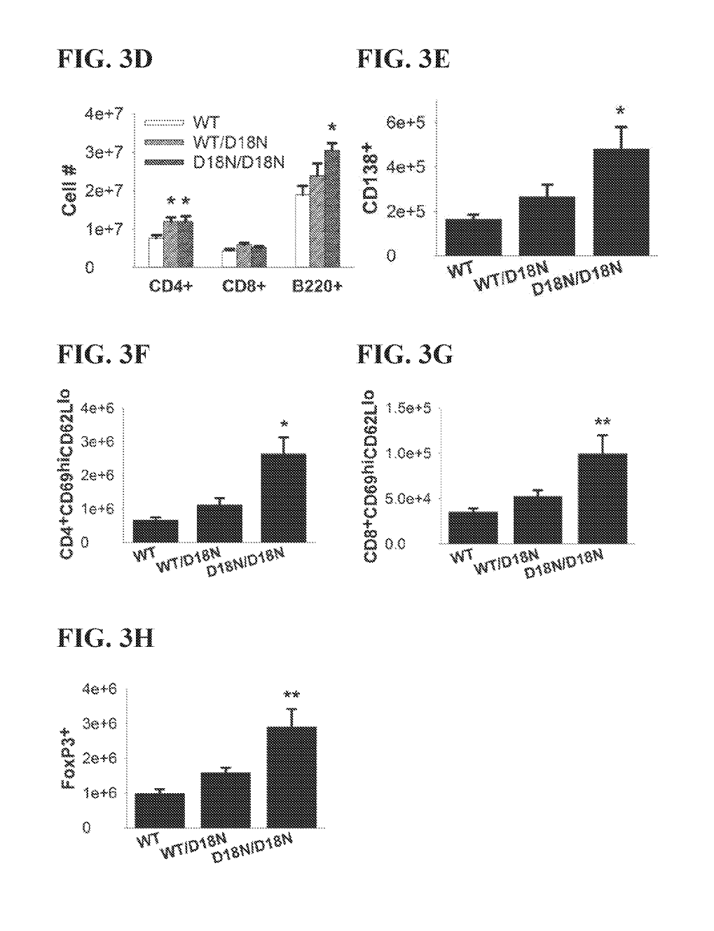

An active inflammatory autoimmune response was present in TREX1^{D18N/D18N} mice that was also detected in TREX1^{WT/D18N} mice as indicated by the overall plasma cell numbers and productivity. The levels of total serum IgG were significantly increased in Trex1^{D18N/D18N} mice compared to TREX1^{WT/WT} mice (FIG. 3A). To determine if dsDNA was a major antigen we measured serum total α-dsDNA antibody levels and found significantly increased levels in TREX1^{D18N/D18N} mice compared to TREX1^{WT/WT} mice (FIG. 3B). Furthermore, immunofluorescence of frozen kidney sections revealed immunocomplexes in glomeruli of TREX1^{D18N/D18N} mice containing IgG (FIG. 3C). To characterize the splenic immune cell population, we labeled splenocytes with markers for T and B cells. Consistent with histologic findings, spleens of TREX1^{D18N/D18N} mice contained increased B220⁺ B cells (FIG. 3D) and CD138⁺ plasma cells (FIG. 3E). Although the number of splenic CD8⁺ T cells was similar between WT and mutant mice, the number of CD4⁺ cells in the spleen of both TREX1^{WT/D18N} and TREX1^{D18N/D18N} mice was increased. Additionally, both CD4⁺ (FIG. 3F) and CD8⁺ (FIG. 3G) splenocytes of TREX1^{D18N/D18N} mice had upregulated expression of the activation marker CD69 demonstrating that cells from mutant mice are more activated and thus more competent at effector functions such as cytokine production and cell killing. Finally, we found that the number of splenic T_{reg} cells was increased in TREX1^{D18N/D18N} mice (FIG. 3H). The Increase in T_{reg} cells could indicate a compensatory response to chronic, uncontrolled inflammation or could indicate that the T_{reg} cells present have decreased function. The functionality of adaptive immune cells including T_{reg} cells will be explored in future work.

TREX1 degradation of dsDNA is key to prevent inappropriate immune activation. The TREX1 D18N-dsDNA structure and the phenotypic characteristics of the TREX1 D18N mouse indicate that TREX1 degrades dsDNA preventing this polynucleotide from acting as an autoantigen in the mouse, and most likely in humans, to inappropriately activate the immune system. Structure and biochemical analyses of TREX1-disease causing mutants identified key amino acids positioned adjacent to the active sites, indicating an extended DNA polynucleotide interaction (8, 20-23). The TREX1 D 18N-dsDNA structure reveals direct contacts with the DNA duplex on the substrate and non-substrate strands. Additionally, important insight into the base-flipping mechanism by which TREX1 separates the DNA strands to efficiently degrade the polynucleotide is learned. Expression of the TREX1 D18N mutant enzyme in mice causes spontaneous autoimmunity and our findings support the idea that failure to appropriately degrade dsDNA is the cause of disease in TREX1^{D18N/D18N} mice due to persistent polynucleotide sensing and immune activation. Genetic studies in humans are revealing mutations in key DNA metabolism enzymes, such as DNA polymerase β (43), that cause autoimmune pathology resembling Lupus when expressed in mice. To our knowledge, expression of the TREX1 D18N allele represents the first time a monogenic form of Lupus in humans has been reproduced by the same genetic change in the mouse. This mutant mouse strain will be a useful tool to further delineate the pathogenic mechanisms of TREX1-mediated autoimmunity specifically as well as the pathogenesis of nucleic-acid mediated autoimmune disease more broadly.

### MATERIALS AND METHODS

Structure Determination. The mouse recombinant Trex1 enzyme (amino acids 1-242) was prepared and crystallized with a dsDNA oligonucleotide. The X-ray data were collected and processed as described further below.

Animals. TREX1 D18N mutant mice were generated on a 129S6/SvEvTac background using an allelic replacement strategy as shown in FIG. 4 and further described below. Transmission of the TREX1 D18N allele was confirmed by sequencing of tail DNA. Males and females exhibited a similar phenotype, so both sexes were used in these studies. All experiments were performed in accordance with the guidelines set forth by the Institutional Animal Care and Use Committee at Wake Forest Baptist Medical Center.

Immunofluorescence. Frozen sections were incubated with antibody to mouse IgG (goat α-mouse IgG Alexa Fluor 488, Abcam), washed three times in PBS, then mounted with Fluoroshield Mounting Medium with DAPI (Abeam). See below for details.

Total IgG and dsDNA antibody ELISA. A total of 4-5 animals (6 mo. old) of each sex and genotype were included in two independent experiments Total IgG ELISA was performed according to the Mouse IgG ELISA Kit (Alpha Diagnostic International, TX, USA) protocol. Total anti-dsDNA antibody ELISA was performed according to the Mouse anti-dsDNA Antibodies Total Ig ELISA Kit (Alpha Diagnostics International). Absorbance at 450 nm was obtained using a Tecan Safire 2 spectrophotometer (Mannedorf, Switzerland) and Tecan Magellan software.

Enzyme Preparation. The mouse recombinant Trex1 enzyme (amino acids 1 -242) was expressed in bacteria and purified as stable homodimers as described (Lehtinen D A et al., J Biol Chem 283(46):31649-31656 (2008); de Silva U, et al. J. Biol. Chem. 282(14):10537-10543 (2007)). Protein concentrations were determined by A₂₈₀ using the molar extinction coefficient for TREX1 protomer ε=23,950 M⁻¹ cm⁻¹.

Protein Crystallization and X-ray Data Collection. The TREX1 D18N mutant was crystallized using the sitting drop vapor diffusion technique. The protein was dialyzed in 20 mM MES (pH 6.5), 50 mM NaCl and concentrated to 10 mg/mL. The pseudo-palindromic oligonucleotide DNA used for crystallization (5′-TCACGTGCTGACGTCAGCACGACG-3′ (SEQ ID NO:1, Operon)) was self-annealed in buffer consisting of 20 mM NaCl, 5 mM MgCl₂, and 5 mM MES, pH 6.5. The complex was formed by incubating dsDNA with the protein in a 1:1 ratio and 5 mM magnesium chloride. A volume of 1 μl protein complex at 4 mg/ml TREX1 was mixed with an equal volume of reservoir solution and placed on a bridge above 500 μl of the reservoir solution. Optimized crystals of the TREX1 D18N mutant grew in 0.1 M sodium acetate and 9% PEG 4000 at 25° C. Prior to data collection all crystals were immersed in reservoir solution containing 10% oil (1:1 mineral oil and pantone-N) in preparation for cryo-cooling. Crystals were mounted on a nylon loop and flash cooled to 100 K in a stream of liquid nitrogen.

Phasing and Refinement. The X-ray data were collected using CuKα radiation on a MicroMax 007 generator and a Saturn 92 CCD detector (Rigaku). Intensity data were processed using the programs d*TREK (Pflugrath J W, Acta Crystallographica Section D-Biological Crystallography 55:1718-1725 (1999)). The TREX1 D18N mutant in complex with dsDNA belongs to the P21 spacegroup (data not shown). Phases for the data were obtained by maximum likelihood molecular replacement using the program PHASER (McCoy A J, et al. Journal of applied crystallography 40(Pt 4):658-674 (2007)) and the TREX1 dimer (PDB ID: 2OA8), including only a single monomer as the search model (protein only). The model was built in the program COOT (Emsley P & Cowtan K Acta Crystallographica Section D-Biological Crystallography 60:2126-2132 (2004)) following composite omit procedures and the structures refined using the programs Refmac5 (Murshudov G N et al., Acta Crystallographica Section D-Biological Crystallography 53:240-255 (1997)), and Phenix.refine (Adams P D, et al. Acta Crystallogr D Biol Crystallogr 58(Pt 11):1948-1954 (2002)). Translation/libration/screw (TLS) refinement was utilized to independently define subgroups and to further refine their directions of movement as individual rigid bodies (Painter J & Merritt E A, Acta Crystallogr D Biol Crystallogr 62(Pt 4):439-450 (2006)). The inspection of clashes and stereochemical parameters was carried out using the program PDB_REDO (Joosten R P et al., IUCrJ 1(Pt 4):213-220 (2014)). The all-atom clashscore is 4.04 and Ramachandran plot shows 97% residues in favored regions and 3% in allowed regions. All structure figures were generated in the program Pymol (Schrodinger, LLC).

Animals. TREX1 D18N mutant mice were generated on a 129S6/SvEvTac background using an allelic replacement strategy as shown in FIG. 4 (Taconic, NY, USA). Briefly, the TREX1 D18N targeting vector was modified by site directed mutagenesis. Embryonic stem cell clones that underwent homologous recombination were selected for expression of the NEO cassette and screened for expression of the mutant allele. Positive D18N clones were expanded and injected into blastocysts. Chimeric mice were bred to Cre deleters to remove the NEO cassette. Transmission of the TREX1 D18N allele was continued by sequencing of tail DNA. Males and females exhibited a similar phenotype, so both sexes were used in these studies. All experiments were performed in accordance with the guidelines set forth by the Institutional Animal Care and Use Committee at Wake Forest Baptist Medical Center.

Genotyping. 1-2 mm tail snips were collected from weanling mice. Genomic DNA was isolated according to the DNeasy kit (Qiagen, MD, USA) protocol and the TREX1 gene was amplified using the following primers:

(SEQ ID NO: 2)
5′ CCTGCTGCTACTCATTACCCCATC 3′
(SEQ ID NO: 3)
5′ CCTACTCCATGTCAGGGAGAGAGGA 3′

OneTaq® DNA Polymerase (New England Biolabs, MA, USA) was used for DNA amplification. Thermocycler conditions were as follows: 94° C. 3 min, (94° C. 30 sec, 54° C. 20 sec, 68° C. 90 sec)×35 cycles. PCR products were resolved on a 1% agarose gel. Bands of the appropriate size (1 KB) were excised and DNA was extracted using a QIAquick Gel Extraction Kit (Qiagen). Sequencing of gel extracted DNA was performed by Genewiz, Inc (NC, USA) using the following primer:

(SEQ ID NO: 4)
5′ ACAGCATCGCTGCCCTAAAG 3′

Expression of TREX1 alleles by RT-PCR. The TREX1 transcript was detected using cDNA prepared from mouse liver total RNA and the following primers:

(SEQ ID NO: 5)
5′ AGGGACAGGGCAGACCAAGAA 3′
(SEQ ID NO: 6)
5′ ACCGGAGTGGACCCGTCATT 3′

Q5® Hot Start High-fidelity 2x Master Mix (New England Biolabs, MA, USA) was used for cDNA amplification. Thermocycler conditions were as follows: 98° C. 2 min, (98° C. 10 sec, 69° C. 20 sec, 72° C. 40 sec)×35 cycles, 72° C. 2 min. PCR products were resolved on a 1% agarose gel.

TREX1 protein purification. Thymus, salivary gland, cervical lymph nodes, heart, liver, kidney, spleen, and brain were pooled from 8 WT and 8 TREX1^{D18N/D18N} male and female mice. Tissues were stored at −80° C. until use and samples were kept on ice or at 4° C. throughout the procedure. Tissue pools were suspended in lysis buffer (50 mM Tris pH 8.2, 1 mM DTT, 1 mM EDTA, 10% glycerol, 10 μg/ml BSA, 0.1 M NaCl, and Complete Protease Inhibitor Cocktail (Roche, Basel, Switzerland)) then disrupted using a dounce homogenizer. Homogenates were centrifuged for 20 minutes at 10,000 rpm (12,000×g). Protamine sulfate (0.12% final concentration) was added to supernatants. Samples were incubated for 5 min then centrifuged for 10 min at 16,000 rpm (30,000×g). Supernatants were collected and dialyzed overnight against dialysis buffer (50 mM Tris pH 8.2, 1 mM DTT, 1 mM EDTA, 10% glycerol, and 0.1 M NaCl). Dialyzed samples were centrifuged for 30 min at 20,000 rpm (48,000×g). Supernatants were loaded onto a single-stranded DNA cellulose (Sigma, MO, USA) column that had been equilibrated overnight in dialysis buffer. The column was washed with dialysis buffer containing 10 μg/ml BSA then with the same buffer containing 0.2 M NaCl then 0.5 M NaCl. Bound proteins were step-eluted with buffer containing 2 M NaCl. Fractions collected during wash and elution steps were dialyzed for 3 hours against dialysis buffer. Concentrated samples were used in western blots and diluted samples were used in exonuclease assays.

TREX1 western blot. Proteins were separated by SDS-PAGE than transferred to a nitrocellulose membrane (Life Technologies, CA, USA). Membranes were blocked with TBS containing 0.1% Tween 20 (TBST) and 5% milk powder then incubated overnight at 4° C. with polyclonal rabbit a-mouse TREX1 antibody diluted 1:100 in TBST. After washing in TBST, membranes were incubated for 1 hr at room temperature with HRP-conjugated anti-rabbit IgG (GE Healthcare, Buckinghamshire, UK). After washing in TBST, bound secondary antibody was visualized by enhanced chemiluminescence (Western Lightening Plus ECL, PerkinElmer, Inc, MA, USA). For generation of the rabbit a-mouse TREX1 antibody, Trex 1 enzyme was recombinately expressed in E. coli and purified by ssDNA cellulose chromatography (Perrino F W, et al., Cell Biochem Biophys 30:331-352 (1999)). Polyclonal antibody was generated to purified Trex 1 enzyme by Rockland, Inc. (PA, USA).

ssDNA exonuclease assay. The exonuclease assays contained 20 mM Tris pH 7.5, 5 mM MgCl₂, 2 mM DTT, 100 μg/ml BSA, 50 nM fluorescein-labeled 30-mer oligonucleotide (Operon, AL, USA), and Trex1 enzyme. Reactions were incubated at 25° C. for 15 min, quenched by the addition of 3 volumes of cold ethanol, and dried in vacuo. The reaction products were resuspended in 4 μl of formamide and separated on 23% denaturing polyacrylamide gels. Fluorescently labeled bands were visualized using a Storm PhosphorImager (GE Healthcare, Buckinghamshire, UK).

qPCR. 100 ng/μl cDNA was added to TaqMan Universal PCR Master Mix and TaqMan assay for TREX1 or Gapdh. Reactions were performed using an Applied Biosystems 7500 Real-time PCR system. Data were analyzed using Applied Biosystems 7500 software v2.0.5. The ΔΔCt method was used to normalize TREX1 expression to Gapdh expression. The level of TREX1 expression in WT liver was set at 1. Data were collected from 3 male and 3 female TREX1^{WT/WT} and TREX1^{D18N/D18N} mice in two independent experiments.

Histology. Tissues were collected from 3-8 mice of each sex and genotype at multiple time points (3 wks., 2 mo., 4 mo., 6 mo.), fixed in 10% neutral buffered formalin for 24-48 hours, decalcified in 0.35 M EDTA if indicated, then processed routinely. Paraffin embedded tissues were sectioned at 5 μm then stained with hematoxylin and eosin (H&E). H&E stained slides were examined and scored by a veterinary pathologist. Lesions were scored as outlined in Tables 2-5.

TABLE 2
Histopathology scoring of renal lesions. Kidneys from 8-10 6 mo. old
mice of each genotype were scored for (A), glomerular lesions; (B),
tubular lesions; and (C), inflammation. Scores from were combined
to determine the overall score reported in FIG. 2I.

| | |
|---|---|
| Score | Description |

A

| | |
|---|---|
| 0 | none |
| 1 | Mild proliferation of mesangial cells and/or membrane thickening |
| | in <50% of glomeruli |
| 2 | Mild proliferation of mesangial cells and/or membrane thickening |
| | in >50% of glomeruli |
| 3 | Moderate proliferation of mesangial cells and/or membrane |
| | thickening in <50% of glomeruli |
| 4 | Moderate proliferation of mesangial cells and/or membrane |
| | thickening in >50% of glomeruli |
| 5 | Severe proliferation of mesangial cells and/or membrane |
| | thickening in <50% of glomeruli |
| 6 | Severe proliferation of mesangial cells and/or membrane |
| | thickening in >50% of glomeruli |
| 7 | Severe proliferation of mesangial cells and/or membrane |
| | thickening in <50% of glomeruli + glomerulosclerosis |
| | in <50% of glomeruli |
| 8 | Severe proliferation of mesangial cells and/or membrane |
| | thickening in >50% of glomeruli + glomerulosclerosis |
| | in >50% of glomeruli |

B

| | |
|---|---|
| 0 | normal |
| 1 | degeneration/regeneration of 1-25% of tubules +/− proteinosis |
| 2 | degeneration/regeneration of 26-75% of tubules +/− proteinosis |
| 3 | degeneration/regeneration of 76-100% of tubules +/− proteinosis |

C

| | |
|---|---|
| 0 | None |
| 1 | Minimal |
| 2 | Mild |
| 3 | Moderate |
| 4 | Severe |
| 5 | Severe with fibrosis |

TABLE 3
Inflammation scoring. Tissues from 8-10 6 mo. old mice of
each genotype were scored for inflammation. Each
organ received an individual score.

| Score | Description |
|---|---|
| 0 | Normal |
| 1 | Minimal |
| 2 | Mild |
| 3 | Moderate |
| 4 | Severe |
| 5 | Severe with extensive tissue destruction/necrosis |

TABLE 4
Lymphoid hyperplasia scoring. Tissues from 8-10 6 mo. old mice
of each genotype were scored for lymphoid hyperplasia.

| Score | Description |
|---|---|
| 0 | none |
| 1 | mild (follicles mildly enlarged and/or mildly increases number of |
| | germinal centers) |
| 2 | moderate (follicles moderately enlarged with less distinct zones |
| | and/or moderate increase in number of germinal centers) |
| 3 | severe (follicles enlarged and coalescing, zones indistinct and/or |
| | marked increase in number of germinal centers) |

TABLE 5
Vasculitis scoring. Tissues from 8-10 6 mo. old mice of each genotype
were scored for vasculitis. Tissues were scored individually. A final
score was determined by adding scores of all affected tissues.

| Score | Description |
|---|---|
| 0 | None |
| 1 | One vessel |
| 2 | Two or more vessels |
| 3 | One or more vessels with extensive tissue destruction/necrosis |

Immunofluorescence. Tissues were frozen in OCT (Sakura Finetek, CA, USA) then stored at −80° C. until sectioning. Tissues were sectioned at 5 μm on a Microm cryostat 525 (Thermo Scientific, MA, USA). Sections warmed to room temperature were fixed for 5 min in ice-cold acetone then washed twice in PBS. Tissues were blocked for 1 hr in PBS containing 1% BSA then washed twice in PBS. Tissues were incubated overnight at 4° C. with antibody to mouse IgG (goat a-mouse IgG Alexa Fluor 488, Abeam) diluted 1:1000 in PBS. Tissues were washed three times in PBS then were mounted with Fluoroshield Mounting Medium with DAPI (Abeam).

Flow cytometry. Spleens collected in two independent experiments from 8 total 4-5 month old female mice of each genotype were mechanically disrupted on a wire mesh screen into RPMI 1640 (Hyclone, UT, USA) supplemented with 10% heat-inactivated FCS (Hyclone), L-glutamine (Hyclone), penicillin-streptomycin (Cellgro, VA, USA), and β-mercaptoethanol (Gibco, NY, USA) (cRPMI). Red blood cells were lysed for 1 minute in ACK buffer (Lonza, Basel, Switzerland). Samples were resuspended in cRPMI and cell counts were performed by hemocytometer. For surface staining, samples were incubated with antibody diluted 1:100 in PBS with 2% FCS for 1 hr at 4° C. For T regulatory cell enumeration, cells were treated according to the Mouse T Regulatory Cell Staining Kit (E Biosciences, Calif., USA) protocol. Samples were acquired on a CANTO II instrument (BD Biosciences, Calif., USA) and data were analyzed using FloJo software (TreeStar, Oreg., USA). The following antibodies (all from BD Biosciences) were used for surface staining: rat α-mouse CD4-PE, rat α-mouse CD8-PerCP, rat α-mouse B220-APC, rat α-mouse CD138-PE, rat α-mouse CD69-FITC, rat α-mouse CD62L-AP-Cy7.

Statistical analysis. Data were expresses as the mean±SEM. Student's t-test was used to compare two independent data sets. Two-way ANOVA was used for multiple comparisons. Differences between groups were determined to be significant when p<0.05. All statistical analyses were performed using SigmaPlot 12.5 software (CA, USA).

### REFERENCES

1. Lindahl T, Gally J A, & Edelman G M (1969) Properties of Deoxyribonuclease III from Mammalian Tissues. J. Biol. Chem. 244:5014-5019.
2. Perrino F W, Miller H, & Ealey K A (1994) Identification of a 3′-->5′-exonuclease that removes cytosine arabinoside monophosphate from 3′ termini of DNA. J. Biol. Chem. 269:16357-16363.
3. Perrino F W, Mazur D J, Ward H, & Harvey S (1999) Exonucleases and the incorporation of aranucleotides into DNA. Cell Biochem Biophys 30:331-352.
4. Hoss M, et al. (1999) A human DNA editing enzyme homologous to the Escherichia coli DnaQ/MutD protein. EMBO J. 18(13):3868-3875.
5. Mazur D J & Perrino F W (1999) Identification and expression of the TREX1 and TREX2 cDNA sequences encoding mammalian 3′-->5′ exonucleases. J Biol Chem 274(28):19655-19660.
6. Mazur D J & Perrino F W (2001) Structure and expression of the TREX1 and TREX2 3′-->5′ exonuclease genes. J Biol Chem 276(18):14718-14727.
7. Mazur D J & Perrino F W (2001) Excision of 3′ termini by the Trex1 and TREX2 3′-->5′ exonucleases. Characterization of the recombinant proteins. J Biol Chem 276(20):17022-17029.
8. de Silva U, et al. (2007) The crystal structure of TREX1 explains the 3′ nucleotide specificity and reveals a polyproline II helix for protein partnering. J. Biol. Chem. 282(14):10537-10543.
9. Richards A, et al. (2007) C-terminal truncations in human 3∝-5′ DNA exonuclease TREX1 cause autosomal dominant retinal vasculopathy with cerebral leukodystrophy. Nat Genet 39(9):1068-1070.
10. Orebaugh C D, et al. (2013) The TREX1 C-terminal region controls cellular localization through ubiquitination. J Biol Chem 288(40):28881-28892.
11. Crow Y J, et al. (2006) Mutations in the gene encoding the 3′-5′ DNA exonuclease TREX1 cause Aicardi-Goutieres syndrome at the AGS1 locus. Nature Genetics 38(8):917-920.
12. Lee-Kirsch M A, et al. (2006) Familial chilblain lupus, a monogenic form of cutaneous lupus erythematosus, maps to chromosome 3p. Amer. J Human Genetics 79(4):731-737.
13. Lee-Kirsch M A, et al. (2007) A mutation in TREX1 that impairs susceptibility to granzyme A-mediated cell death underlies familial chilblain lupus. Journal of Molecular Medicine-Jmm 85(5):531-537.
14. Rice G, et al. (2007) Clinical and molecular phenotype of aicardi-goutieres syndrome. Amer. J Human Genetics 81(4):713-725.
15. Crow Y J & Rehwinkel J (2009) Aicardi-Goutieres syndrome and related phenotypes: linking nucleic acid metabolism with autoimmunity. Hum Mol Genet 18(R2):R130-136.
16. Gunther C, Berndt N, Wolf C, & Lee-Kirsch M A (2014) Familial Chilblain Lupus Due to a Novel Mutation in the Exonuclease III Domain of 3′ Repair Exonuclease 1 (TREX1). JAMA dermatology.
17. Lee-Kirsch M A, et al. (2007) Mutations in the gene encoding the 3′-5′ DNA exonuclease TREX1 are associated with systemic lupus erythematosus. Nature Genetics 39(9):1065-1067.
18. de Vries B, et al. (2010) TREX1 Gene Variant in Neuropsychiatric Systemic Lupus Erythematosus. Ann Rheum Dis 69(10): 1886-1887.
19. Namjou B, et al. (2011) Evaluation of the TREX1 gene in a large multi-ancestral lupus cohort. Genes Immun 12(4):270-279.
20. Bailey S L, Harvey S, Perrino F W, & Hollis T (2012) Defects in DNA degradation revealed in crystal structures of TREX1 exonuclease mutations linked to autoimmune disease. DNA Repair (Amst) 11 (1):65-73 .
21. Fye J M, Coffin S R, Orebaugh C D, Hollis T, & Perrino F W (2014) The Arg-62 Residues of the TREX1 Exonuclease Act Across the Dimer Interface Contributing to Catalysis in the Opposing Protomers. J Biol Chem 289(16):11556-11565.
22. Orebaugh C D, Fye J M, Harvey S, Hollis T, & Perrino F W (2011) The TREX1 Exonuclease R114H Mutation in Aicardi-Goutieres Syndrome and Lupus Reveals Dimeric Structure Requirements for DNA Degradation Activity. J Biol Chem 286(46):40246-40254.
23. Fye J M, Orebaugh C D, Coffin S R, Hollis T, & Perrino F W (2011) Dominant mutation of the TREX1 exonuclease gene in lupus and Aicardi-Goutieres syndrome. J Biol Chem 286(37):32373-32382.
24. Lehtinen D A, Harvey S, Mulcahy M J, Hollis T, & Perrino F W (2008) The TREX1 double-stranded DNA degradation activity is defective in dominant mutations associated with autoimmune disease. J Biol Chem 283(46):31649-31656.
25. Morita M, et al. (2004) Gene-targeted mice lacking the Trex 1 (DNaseIII) 3′-5′ DNA exonuclease develop inflammatory myocarditis. Mol. Cell. Biol. 24(15):6719-6727.
26. Chowdhury D, et al. (2006) The exonuclease TREX1 is in the SET complex and acts in concert with NM23-H1 to degrade DNA during granzyme A-mediated cell death. Mol Cell 23(1):133-142.
27. Lindahl T, Barnes D E, Yang Y G, & Robins P (2009) Biochemical properties of mammalian TREX1 and its association with DNA replication and inherited inflammatory disease. Biochem Soc Trans 37(Pt 3):535-538.
28. Yang Y G, Lindahl T, & Barnes D E (2007) Trex 1 exonuclease degrades ssDNA to prevent chronic checkpoint activation and autoimmune disease. Cell 131(5):873-886.
29. Ablasser A, et al. (2014) TREX1 deficiency triggers cell-autonomous immunity in a cGAS-dependent manner. J Immunol 192(12):5993-5997.
30. Gall A, et al. (2012) Autoimmunity Initiates in Nonhematopoietic Cells and Progresses via Lymphocytes in an Interferon-Dependent Autoimmune Disease. Immunity 36(1):120-131.
31. Stetson D B, Ko J S, Heidmann T, & Medzhitov R (2008) Trexl prevents cell-intrinsic initiation of autoimmunity. Cell 134(4):587-598.
32. Sun L, Wu J, Du F, Chen X, & Chen Z J (2013) Cyclic GMP-AMP synthase is a cytosolic DNA sensor that activates the type I interferon pathway. Science 339(6121):786-791.
33. Volkman H E & Stetson D B (2014) The enemy within: endogenous retroelements and autoimmune disease. Nat Immunol 15(5):415-422.
34. Yan N, Regalado-Magdos A D, Stiggelbout B, Lee-Kirsch M A, & Lieberman J (2010) The cytosolic exonuclease TREX1 inhibits the innate immune response to human immunodeficiency virus type 1. Nat Immunol 11(11):1005-1013.
35. Tsutakawa S E, Lafrance-Vanasse J, & TaMer J A (2014) The cutting edges in DNA repair, licensing, and fidelity: DNA and RNA repair nucleases sculpt DNA to measure twice, cut once. DNA Repair (Amst) 19:95-107.
36. Tsutakawa S E, et al. (2011) Human flap endonuclease structures, DNA double-base flipping, and a unified understanding of the FEN1 superfamily. Cell 145(2):198-211.
37. Williams R S, et al. (2008) Mre11 dimers coordinate DNA end bridging and nuclease processing in double-strand-break repair. Cell 135(1):97-109.
38. Hsiao Y Y, Duh Y, Chen Y P, Wang Y T, & Yuan H S (2012) How an exonuclease decides where to stop in trimming of nucleic acids: crystal structures of RNase T-product complexes. Nucleic Acids Res 40(16):8144-8154.
39. Pereira-Lopes S, et al. (2013) The exonuclease Trex1 restrains macrophage proinflammatory activation. J Immunol 191(12):6128-6135.
40. Pathak S & Mohan C (2011) Cellular and molecular pathogenesis of systemic lupus erythematosus: lessons from animal models. Arthritis Res Ther 13(5):241.
41. Perry D, Sang A, Yin Y, Zheng Y Y, & Morel L (2011) Murine models of systemic lupus erythematosus. J Biomed Biotechnol 2011:271694.
42. Rottman J B & Willis C R (2010) Mouse models of systemic lupus erythematosus reveal a complex pathogenesis. Vet Pathol 47(4):664-676.
43. Senejani A G, et al. (2014) Mutation of POLB causes lupus in mice. Cell Rep 6(1):1-8.

The foregoing is illustrative of the present invention, and is not to be construed as limiting thereof. The invention is defined by the following claims, with equivalents of the claims to be included therein.

1. A recombinant non-human mammal, wherein the mammal comprises in its genome a recombinant nucleic acid encoding a mutant three prime exonuclease 1 (TREX1), and which mammal expresses said mutant TREX1.

2. The mammal of claim 1, wherein said mammal expresses auto-antibodies to double stranded DNA.

3. The mammal of claim 1, wherein said mammal exhibits systemic inflammation, lymphoid hyperplasia, vasculitis, and/or kidney disease (e.g., deposition of immune complexes in the kidneys).

4. The mammal of claim 1, wherein said recombinant nucleic acid is operatively associated with an endogenous TREX1 promoter.

5. The mammal of claim 1, wherein said mammal is a rodent or primate.

6. The mammal of claim 1, wherein said mammal is a mouse.

7. The mammal of claim 1, wherein said mammal is homozygous or heterozygous for said mutant TREX1.

8. The mammal of claim 1, wherein said mutant TREX1 contains a substitution mutation.

9. The mammal of claim 8, wherein said substitution mutation is a D18N substitution mutation.

10. The mammal of claim 8, subject to the proviso that said substitution mutation is not a D18N substitution mutation.

11. The mammal of claim 8, wherein said substitution mutation is a D18H substitution mutation.

12. The mammal of claim 8, wherein said substitution mutation is selected from the group consisting of D200N, D200H, D200A, R114H, T303P, Y305C, P290L, and G306A substitution mutations.

13. The mammal of claim 8, wherein said substitution mutation is selected from the group consisting of T13N, T32R, K66R, L92Q, R97H, V122A, R128H, P132A, A158V, L162P, R185C, H195Q, H195Y, E198K, V201D, V201N, D220G, A223T, G227S, R240S, and A247P substitution mutations.

14. A method of identifying a candidate compound for the treatment of autoimmune disease, comprising:
(a) providing a transgenic non-human mammal of claim 1;
(b) administering a test substance to said non-human mammal; and
(c) determining whether said test substance reduces at least one indicia of autoimmune disease in said mammal, wherein a reduction in said at least one indicia indicates said test substance is a candidate compound for the treatment of autoimmune disease.

15. The method of claim 14, wherein said determining step is carried out by:
(a) measuring said at least one indicia in said mammal before said administering step, measuring said at least one indicia in said mammal after said administering step, and comparing the two; and/or
(b) comparing said at least one indicia in said mammal after said administering step with said at least one indicia in a corresponding transgenic non-human mammal that has not been administered said test substance.

16. The method of claim 14, wherein said at least one indicia is expression of auto-antibodies to double stranded DNA, systemic inflammation, lymphoid hyperplasia, vasculitis, kidney disease, or a combination thereof.

17. The method of claim 14, wherein the autoimmune disease is selected from the group consisting of: systemic lupus erythmatosus (SLE); Aicardi-Goutieres syndrome (AGS); familial chilblain lupus (FCL); STING-associated vasculopathy with onset in infancy (SAVI); Sjögren's syndrome; scleroderma; and retinal vasculopathy with cerebral leukodystrophy (RVCL).

18. The method of claim 14, wherein the test substance binds to and/or inhibits cyclic GMP-AMP synthase (cGAS).

19. The method of claim 14, wherein the test substance binds to and/or inhibits stimulator of interferon genes (STING).

20. The method of claim 14, wherein the test substance binds to and/or inhibits TANK-binding kinase 1 (TBK1) and/or interferon regulatory factor 3 (IRF3).
